(19) United States
(12) Plant Patent Application Publication
(10) Pub. No.: US 2015/0059039 P1
(43) Pub. Date: Feb. 26, 2015

(54) Sedum plant named 'Marina'

(50) ;

(71) Applicant: Terra Nova Nurseries, Inc., Canby, OR (US)

(72) Inventor: Janet N. Egger, Wilsonville, OR (US)

(73) Assignee: Terra Nova Nurseries, Inc., Canby, OR (US)

(21) Appl. No.: 13/987,663

(22) Filed: Aug. 20, 2013

(51) Int. Cl. A01H 5/00

(52) U.S. Cl. PLT/479

(57) ABSTRACT

A new and distinct hybrid of Sedum plant substantially as shown and described, characterized by rose red flowers from pink buds, blue green leaves tinted lavender in the summer, rose pink stems, red carpels when the seed is developing, a dense, spreading habit, and excellent vigor.

### BOTANICAL DENOMINATION

Sedum telephium (Syn. Hylotelephium telephium)

### VARIETY DESIGNATION

‘Marina’

### BACKGROUND OF THE INVENTION

The present invention relates to a new and distinct cultivar of Sedum telephium and given the cultivar name of ‘Marina’. Sedum is in the family Crassulaceae. The new cultivar is part of a planned breeding program to make short, compact, multicrown plants. The new cultivar originated from an open pollinated cross of a proprietary seedling, Sedum 26-5.

Compared to the seed parent, Sedum 26-5, the new cultivar has a much better habit and longer, larger leaves.

Compared to Sedum ‘Yellow Xenox’, U.S. Plant Pat. No. 21,528, the new variety is has rose red flowers rather than pink, blue green leaves rather than yellow green, and a wider habit with more crowns from young plants (an advantage to a nursery grower as the plants fill the pots quickly).

This new and unique Sedum is distinguished by:

1. rose red flowers from pink buds,

2. blue green leaves tinted lavender in the summer,

3. rose pink stems,

4. red carpels when the seed is developing,

5. a dense, spreading habit, and

6. excellent vigor.

This new cultivar has been reproduced only by asexual propagation (division and tissue culture). Each of the progeny exhibits identical characteristics to the original plant. Asexual propagation by division and tissue culture using standard micropropagation techniques with terminal and lateral shoots, as done in Canby, Oreg., shows that the foregoing characteristics and distinctions come true to form and are established and transmitted through succeeding propagations. The present invention has not been evaluated under all possible environmental conditions. The phenotype may vary with variations in environment without a change in the genotype of the plant.

### BRIEF DESCRIPTION OF THE DRAWINGS

FIG. 1 shows the foliage of a 3 year old Sedum ‘Marina’ growing in the trial bed in early June in Canby, Oreg.

FIG. 2 shows a close up the inflorescence of Sedum ‘Marina’ in bud in early July.

FIG. 3 shows one year old plants of Sedum ‘Marina’ in bloom in the trial field in late August in Canby, Oreg.

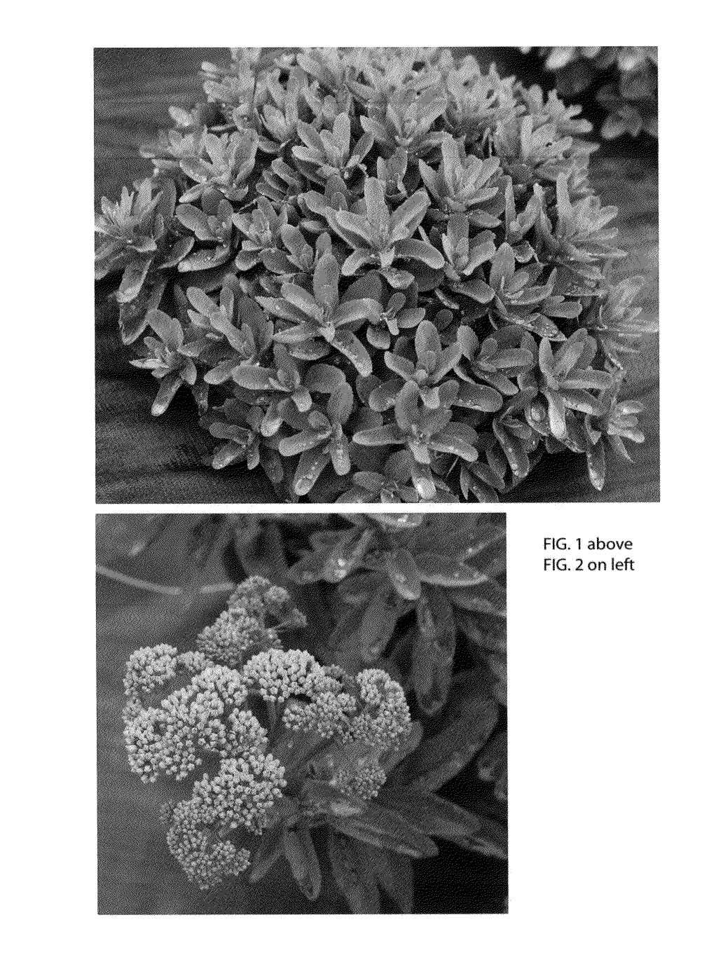

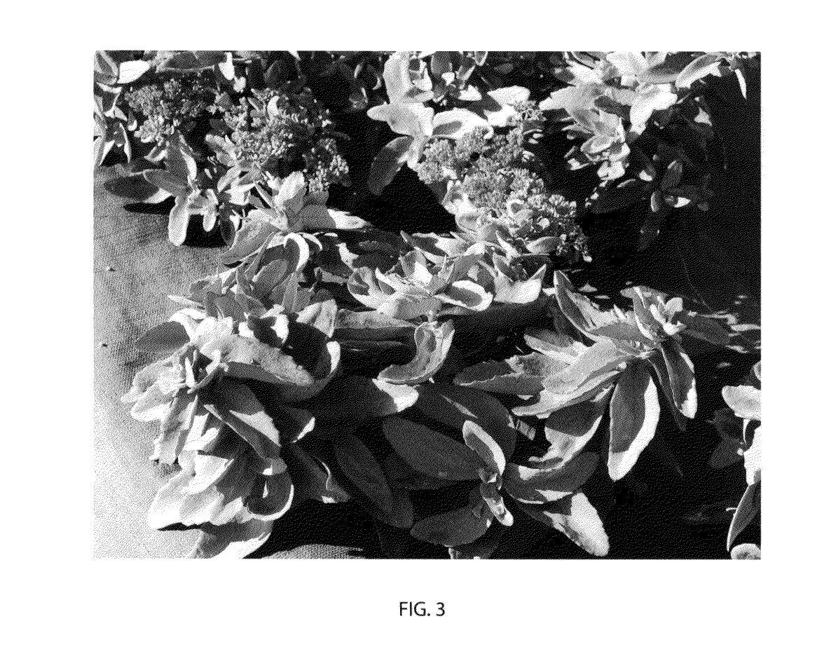

### DETAILED PLANT DESCRIPTION

The following is a detailed description of the new Sedum hybrid based on observations of one year old specimens grown in the trial bed in full sun under typical outdoor conditions in Canby, Oreg. Canby is Zone 8 on the USDA Hardiness map. Temperatures range from a high of 95° F. in August to 32° F. in January. Normal rainfall in Canby is 42.8 inches per year. The color descriptions are all based on The Royal Horticultural Society Colour Chart, 5^{th} edition.
Plant:
   Type.—herbaceous perennial.
   Hardiness.—USDA Zone 4 to 9.
   Size.—10 cm tall from the ground to the base of the inflorescence and 35 cm wide.
   Habit.—low, clumping mound.
   Vigor.—excellent.
Stem:
   Number.—about 6.
   Size.—10 cm long and 7 mm wide.
   Internode length.—2 mm to 20 mm long.
   Color.—Yellow Green 1468 in shade and Greyed Purple 187A in sun.
   Texture.—succulent.
   Surface.—glabrous.
Leaf:
   Type.—simple.
   Arrangement.—opposite.
   Shape.—oblong.
   Apex.—obtuse.
   Base.—obtuse.
   Venation.—pinnate, topside main vein Greyed Green 191A at base, bottom side main vein Yellow Green 148C.
   Margins.—irregularly serrate.
   Blade size.—grows to 10 cm long and 4 cm wide.
   Surface.—glabrous on both sides.
   Texture.—thick, spongy, succulent.
   Petiole.—leaves sessile.
   Leaf color.—topside Greyed Green 189A with purple tints Greyed Purple N187A in summer; bottom side Greyed Green 191A.
Inflorescence:
   Type.—terminal, corymbose, umbel-like.
   Number of flowers.—about 300 per stem (increases with age).
   Pedicels.—2 mm to 3 mm long, 1 mm wide, glabrous, closest to Greyed Green 197A.
   Peduncle.—grows to 2 cm tall and 4 mm wide, Greyed Purple 183A.
   Size.—grows to 10.5 cm deep and 10 cm wide.
   Bloom period.—August through September in Canby, Oreg.
Flower bud:
   Size.—2.5 mm wide, 3 mm deep.
   Description.—ovoid.
   Color.—Greyed Purple 186C.
Flower:
   Type.—actinomorphic.
   Shape.—stellate.
   Size.—4 mm deep and 4 mm wide.
   Corolla description.—4 mm deep and 4 mm wide, 5 petals, each lanceolate, 4 mm long and 1.5 mm wide, margins entire, tip acuminate, glabrous on both sides, top side Greyed Purple 186C on top ⅔ and Greyed Purple 186D on bottom ⅔; bottom side Greyed Purple 186C.
   Calyx description.—2 mm deep and 2.5 mm wide, stellate, 5 lobes, divided to the base, margins entire, tip acute, glabrous, Greyed Green 197A on both sides.
   Stamen description.—10, 3 mm long, filaments 3 mm long and Greyed Purple 187A, anthers sterile and reduced, no pollen.
   Pistil description.—5 separate prominent carpels, each 4 mm deep and 1.5 mm wide, Red Purple 64A, style 1 mm long, stigma and style Greyed Purple 187A.
   Fragrance.—none.
   Lastingness.—each stem lasts up to a month.
Fruit:
   Type.—Ventrally dehiscent follicle, erect, attenuate.
   Fertility.—low.
   Size.—3 mm wide and 3 mm deep.
   Color.—Greyed Purple 187A.
Seed: None seen
Disease and pest resistance or tolerance: Pest and disease resistance are typical for this type of Sedum. No known resistances.

I claim:

1. A new and distinct hybrid of Sedum plant as herein illustrated and described.
